(19) United States
(12) Patent Application Publication
(10) Pub. No.: US 2021/0386501 A1
(43) Pub. Date: Dec. 16, 2021

(54) PATIENT RESPIRATORY ISOLATION SHIELD DEVICES AND METHODS

(71) Applicant: INSPIRE MEDICAL SYSTEMS, INC., Golden Valley, MN (US)

(72) Inventors: John Rondoni, Plymouth, MN (US); Luke Lozier, Shorewood, WI (US); Brian Mullins, Minneapolis, MN (US); Dallas Erdahl, Minneapolis, MN (US); Avery Weigle, St. Paul, MN (US)

(73) Assignee: INSPIRE MEDICAL SYSTEMS, INC., Golden Valley, MN (US)

(21) Appl. No.: 17/342,908

(22) Filed: Jun. 9, 2021

Related U.S. Application Data
(60) Provisional application No. 63/037,162, filed on Jun. 10, 2020.

(51) Int. Cl. A61B 90/00; A61B 1/00

(52) U.S. Cl. CPC A61B 90/05; A61B 1/00142

(57) ABSTRACT

A system and/or method for isolating respiratory excretions from a patient during a medical procedure performed on the airways of a patient. A shield device includes a barrier, one or more support arms, and an access port. The barrier can be a flexible material, and is impervious to air. The support arm(s) maintain the barrier in a deployed state for placement over a patient's head, including the patient's mouth and nose being located within an isolation region established by the barrier. The access port is carried by the barrier and is configured to permit sealed passage of a medical device (e.g., an endoscope) into the isolation region for interfacing with the patient's airway.

### CROSS-REFERENCE TO RELATED APPLICATIONS

This application claims the benefit of the filing date of U.S. Provisional Application Ser. No. 63/037,162, filed Jun. 10, 2020 and entitled “Patient Respiratory Isolation Shield Devices and Methods,” the entire teachings of which are incorporated herein by reference

### BACKGROUND

Various procedures entail placement of an endoscope into the airways of a sedated patient, for example drug-induced sleep endoscopy. During the course of such procedures, it is not uncommon for patients to experience dramatic shifts in airway pressures during one or both of inhalation and exhalation. Further, patients may also have sneezes, coughs, and the like over the course of the particular procedure. These and other circumstances can result in the release or secretion of aerosolized particles or droplets from the patient's respiratory system into the immediately surrounding environment. Care providers and surfaces in this surrounding environment can thus be exposed to infectious agents in the droplets. This possibility can pose substantive risks when performing airway-related medical procedures on patients known or suspected to be suffering from a highly communicable respiratory illness, such as COVID-19.

### BRIEF DESCRIPTION OF THE DRAWINGS

FIG. 1A is a simplified top view of a shield device in accordance with principles of the present disclosure.

FIG. 1B is a side view of the shield device of FIG. 1A.

FIG. 1C is a longitudinal cross-sectional view of the shield device of FIG. 1A, taken along the line 1C-1C.

FIG. 1D is a transverse cross-sectional view of the shield device of FIG. 1A, taken along the line 1D-1D.

FIGS. 2-5 are simplified side views illustrating use of the shield device of FIG. 1A with a patient lying on a support.

FIG. 6A is a top perspective view of a shield device in accordance with principles of the present disclosure.

FIG. 6B is a bottom perspective view of the shield device of FIG. 6A.

FIG. 7A is a flat plan view of a front panel of the shield device of FIG. 6A.

FIG. 7B illustrates a flap component of the shield device of FIG. 6A assembled to the front panel of FIG. 7A.

FIG. 8 is a side view of the shield device of FIG. 6A in a collapsed or storage state.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to shields or protective devices useful with patients undergoing a respiratory endoscopic procedure, such as drug-induced sleep endoscopy. At least some examples may comprise a shield that facilitates insertion of an endoscope or the like into a patient's airway while protecting the health care workers and surfaces in the immediately surrounding environment from exposure to respiratory excretions from the patient, and thus any aerosolized particles or droplets carried by such excretions.

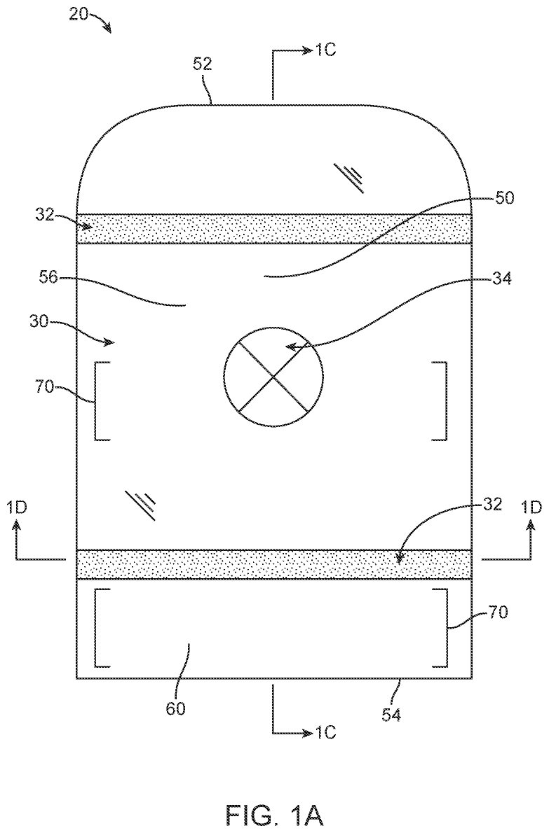

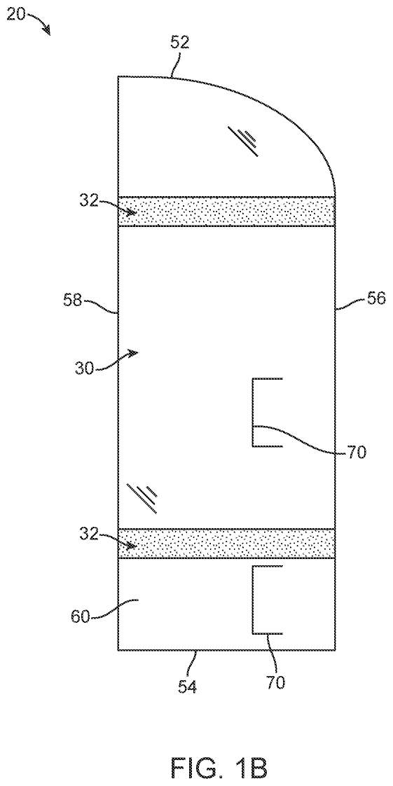

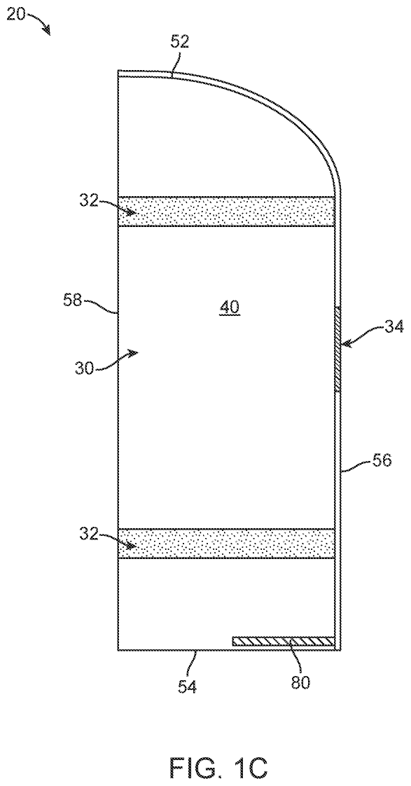

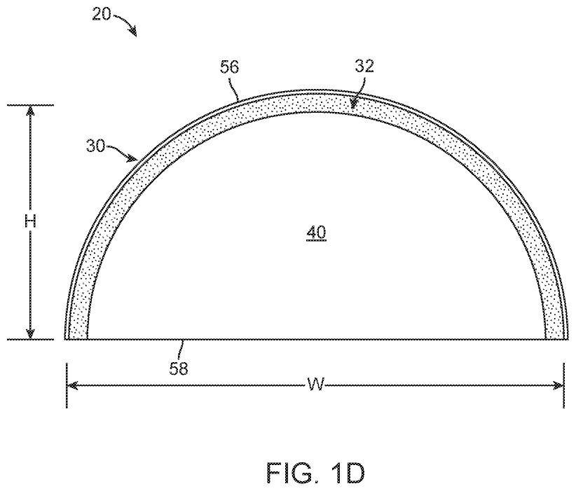

One example of a shield device 20 in accordance with principles of the present disclosure is shown in FIGS. 1A-1D. The shield device 20 includes a barrier 30, one or more support arms 32, and an access port 34. Details on the components are provided below. In general terms, in the deployed state of FIGS. 1A-1D, the support arms 32 retain the barrier 30 in a shape conducive for placement over a head and neck reclined patient, creating an isolation region 40 (referenced generally in FIGS. 1C and 1D) that encompasses at least the patent's mouth and nose. All or virtually all particles, droplets, etc., emanating from the patient's mouth and nose (e.g., respiratory excretions) are contained within the isolation region 40 and are prevented from escaping to the surrounding environment as described below. Finally, the access port 34 facilitates placement of a medical device within the airway of a patient otherwise stationed within the isolation region 40 from an exterior of the shield device 20 in a manner that does not affect containment of respiratory excretions, such as, for example, placement of an endoscope into the patient's nose or mouth. The shield device 20 permits performance of various respiratory-related procedures on a patient, for example drug-induced sleep endoscopy, with minimal or no risk of aerosolized particles or droplets from the patient's respiratory system coming in contact with a care provider or escaping into the surrounding environment.

As a point of reference, in the cross-sections of FIGS. 1C and 1D, a thickness of the barrier 30 is exaggerated for ease of understanding. With this in mind, the barrier 30 can assume various forms, and in some embodiments includes or comprises a flexible sheet of material that is impervious to air and bacterial sub-particles. For example, a material of the barrier 30 can be, or can be akin to, a polymer typically used for surgical gloves, such as nitrile rubber, polyvinyl chloride, neoprene, latex, etc. Alternatively, an appropriate fabric material can be used as or with the barrier 30. Regardless, at least a viewing area 50 (referenced generally in FIG. 1A) of the barrier 30 is formed to be substantially transparent (i.e., within 10% of truly transparent). In other embodiments, an entirety of the barrier 30 can be substantially transparent.

The barrier 30 extends from a top end 52 to a bottom end 54, and defines a front side 56. With reference to a shape of the barrier 30 in the deployed state, the top end 52 can be considered closed, whereas the bottom end 54 is open (i.e., the bottom end 54 is open to the isolation region 40). In some embodiments, a back side 58 of the barrier 30 is open; however, during use the shield device 20 can be arranged over a reclined patient (not shown) with the back side 58 contacting or relatively sealed to the surface supporting the patient, thereby “closing” the back side 58. In other embodiments, the barrier 30 can form part or all of the back side 58 as a more complete structure (e.g., the back side 58 is closed to the isolation region 40) such that the barrier 30 is akin to a bag.

The support arm(s) 32 can assume various forms appropriate for supporting the barrier 30 to the general shape depicted in FIGS. 1A-1D (e.g., the support arm(s) 32 can have the curved or dome-like shape best shown in FIG. 1D). In some embodiments, the support arm(s) 32 can be a strong yet flexible body, such as plastic, fiberglass, aluminum, etc., of a type used with tents, although other material are also acceptable. In some embodiments, the support arm(s) 32 can rigidly maintain the shape of the deployed state. In other embodiments, the support arm(s) 32 can be configured to be collapsible from the deployed state, and readily actuated or shaped by a user to the arrangement of FIGS. 1A-1D. For example, the support arm(s) 32 can include or incorporate a deflection mechanism or biasing device (e.g., a spring or spring mechanism can be incorporated into the support arm 32 that facilitates deployment to, and retention of, the shape of FIGS. 1A-1D). While FIGS. 1A and 1B illustrate two of the support arms 32 located proximate the top end 52 and the bottom end 54, respectively, any other number, either greater or lesser, is also acceptable. In some optional embodiments, at least a bottom segment 60 (referenced generally in FIGS. 1A and 1B) of the barrier 30 is free of the support arms 32 for reasons made clear below.

The support arm(s) 32 can be secured to the barrier 30 in various manners. In some embodiments, the barrier 30 is permanently attached to the support arm(s) 32 (e.g., adhesive, stitching, welding, etc.). In other embodiments, the shield device 20 can be configured such that the barrier 30 is removably connected to the support arm(s) 32. For example, complementary hook-and-loop fastener material strips (e.g., Velcro®) can be supplied with the barrier 30 and the support arm(s) 32. With these and related optional embodiments, the barrier 30 can be considered a one-time or disposable article, whereas the support arm(s) 32 can be sterilized and re-used.

Regardless of an exact construction, a size and shape of the shield device 20 in the deployed state (e.g., a size and shape of the barrier 30 as dictated by the support arms 32) is selected in accordance with human adult form factors, and in particular to receive a patient's head and neck within the isolation region 40. For example, a length of the isolation region 40 (i.e., linear distance from the top end 52 to the bottom end 54) in the deployed state is selected to approximate (e.g., be slightly greater than) the length from the top of the head to the base of the neck of a typical human adult. As identified in FIG. 1D, a height H and width W of the isolation region 40 is sized and shaped to be at least slightly larger than a typical adult human head.

The access port 34 can assume various forms conducive to insertion and removal of a surgical device (e.g., an endoscope) in a sealed (e.g., airtight) manner. For example, the access port 34 can be, or can be akin to, an iris port, including a slit/sealable membrane (e.g., silicone) secured over an opening through a thickness of the barrier 30 by a grommet or similar device. Other constructions are also acceptable. Regardless, the access port 34 is located along the front side 56 of the barrier 30, spaced from the support arms 32. In some embodiments, a location of the access port 34 relative to a length of the barrier 30 (e.g., location between the top end 52 and the bottom end 54) is selected to approximate a likely location of a patient's mouth or nose when stationed within the isolation region 40 for reasons made clear below.

The shield devices of the present disclosure can optionally include one or more additional features. For example, one or more access flaps 70 can be formed through a thickness of the barrier 30. The optional flap(s) 70 are configured to facilitate access to the isolation region 40 by a care giver's hand in a manner that does not compromise an integrity of the isolation region 40 (e.g., when a user's hand is placed through the flap 70, airflow, particles, etc., within the isolation region 40 cannot escape to the external environment in some non-limiting examples). A number, size and location of the flap(s) 70 can vary from the constructions implicated by the views.

As shown in FIG. 1C, the shield device 20 can optionally include a filter or filter media 80. The filter 80 can assume various forms (e.g., a HEPA filter material) and is secured to the barrier 30 at or proximate the bottom end 54. With these and related embodiments, the filter 80 serves to remove entrained particles from airflow within the isolation region 40. In related embodiments, the shield device 20 can form or carry a port or similar airflow connector to a positive or negative pressure source (e.g., a standard hospital suction canister or negative pressure source). The airflow connector can be near or at the filter 80. In other embodiments, the airflow connector can be opposite the filter (e.g., can be formed at or carried by the top end 52). In yet other embodiments, a filter can be provided in tubing to the positive or negative pressure source. With these and similar embodiments, the shield device 20 can optionally further include a pressure gauge that is supported by, for example, the barrier 30 and is open to the isolation region 40. Where so provided, the pressure gauge can visually display a pressure within the isolation region.

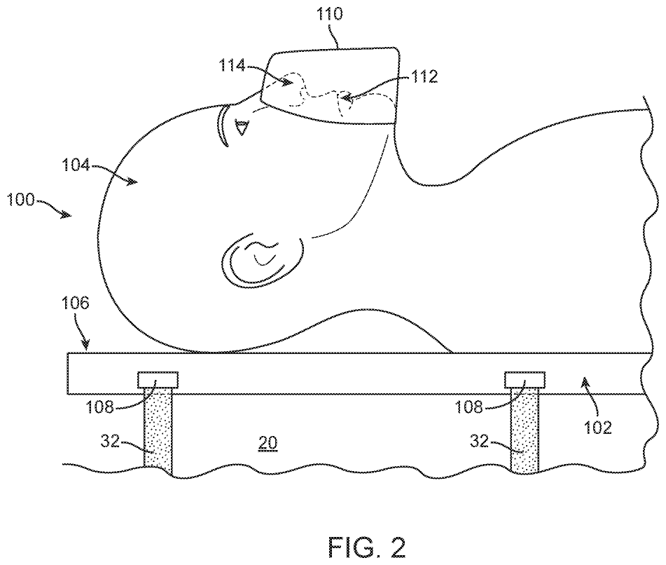

The shield devices of the present disclosure are useful in facilitating performance of a plethora of respiratory-related procedures at a desired location due, at least in part, to a small size or footprint as well as portability and ease of use. By way of non-limiting example, the shield device 20 can be used during performance of a drug-induced sleep endoscopy procedure. Some examples of procedures and methods in accordance with principles of the present disclosure can begin with a patient 100 placed or lying supine on a support 102 (e.g., a bed such as a conventional health care clinic bed or the like) as in FIG. 2, with a head 104 of the patient 100 being supported by a surface 106. The shield device 20 is not yet deployed over the patient 100. In some optional embodiments, the shield device 20 can be mounted or connected to, or carried by, the support 102, for example by hinges 108 as generally reflected by FIG. 2 (e.g., the hinges 108 can be connected to a respective one of the support arms 32). In other embodiments, the shield device 20 can be entirely separate from the support 102 and delivered by a care provider to the patient 100. With these and related embodiments, the shield device 20 can be configured for deployment about the patient's head 104 while the patient is lying on the surface 106; in other embodiments, the shield device 20 can be configured such that patient's head 104 is off of the surface 102 for placement of the shield device 20 (e.g., with embodiments in which the shield device 20 is akin to a bag).

With some procedures, for example drug-induced sleep endoscopy procedures, the patient 100 may be sedated while supine on the surface 106 (e.g., prior to installation of the shield device 20 over the patient 100). The level of sedation can vary as a function of the particular procedure, and can be accomplished with various anesthesia techniques as known in the art. With drug-induced sleep endoscopy procedures, it may be beneficial to minimize the level of pharmacologic sedation such that the patient 100 remains arousable to verbal stimuli (mild sedation). Regardless, while the patient 100 may or may not be wearing a conventional mask 110 over the mouth 112 and nose 114, the shield device 20 is not deployed or installed over the patient 100 as part of the sedation process, allowing the patient 100 to start to or fall asleep without experiencing claustrophobia.

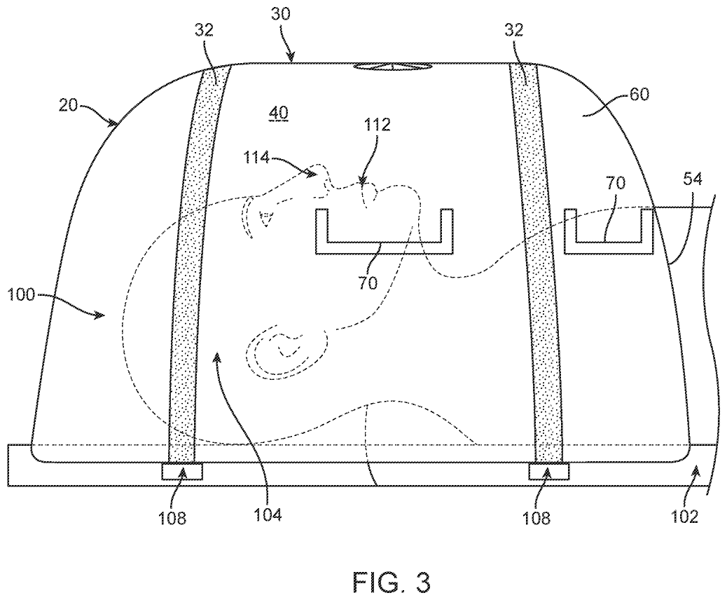

FIG. 3 illustrates a later stage of the procedure, with the shield device 20 now deployed or installed over the patient's head 104 (the mask 110 of FIG. 2 (if used) has been removed). With embodiments in which the shield device 20 is connected to the support 102, the shield device 20 can be pivoted at the hinges 108 (or similar mechanism) to bring the barrier 30 over the patient's head 104. In related embodiments, the hinges 108 can further be slidably connected to the support 102, allowing re-positioning of the support device 20 relative to the patient's mouth 112 and nose 114 prior to or after deployment. In other embodiments, the shield device 20 can be manipulated to the deployed state and then placed about the patient's head 104 (and resting, for example, on the surface 106). Regardless, following deployment or installation of the shield device 20, the patient's mouth 112 and nose 114 reside within the isolation region 40 (referenced generally), with the barrier 30 being at least slightly spaced away from the mouth 112 and nose 114 (e.g., the support arms 32 are sized and shaped so as to maintain the barrier 30 a short distance away from the mouth 112 and nose 114) as well as a remainder of the patient's face. With these and related embodiments, presence of the barrier 30 is less likely to cause feelings of claustrophobia in the patient 100, and the shield device 20 will not overtly arouse the patient 100, negatively affect possible assessment studies being performed on the patient 100, etc. Moreover, where the barrier 30 is substantially transparent, the patient 100 is even less likely to experience feelings of claustrophobia.

The bottom segment 60 of the barrier 30 may pucker or droop towards the patient 100 to provide a partial seal for the isolation region 40. While the shield device 20 is generally illustrated as being sized such that the bottom end 54 is located approximately below the patient's neck/shoulders, other sizes (and thus locations of the bottom end 54) are also acceptable. For example, the shield device 20 can be sized and shaped such that the bottom end 54 is aligned with the patient's chest. In yet other embodiments, the shield devices of the present disclosure can optionally incorporate features that provide a more robust connection to the patient's body, for example relative to the patient's neck and/or arms. For example, the shield device can be akin to a turtle neck, can include stretchy fabric that can easily be adjusted with arm loops, can include or carry elastic or Velcro®, etc.

With the arrangement of FIG. 3, virtually all, if not all, particles, droplets, etc., generated by the patient's respiratory system and emanating from the mouth 112 and/or nose 114 are contained within the isolation region 40. The shield device 20 thus creates a robust barrier, reducing distribution of respiratory droplets that could otherwise escape outside the contained space around the patient 100. The patient 100 can sneeze or cough without violating the seal. Particles, droplets, etc., can be contained within the isolation region 40 throughout the procedure (e.g., collecting on an inner surface of the barrier 30). In other optional embodiments, airflow can be provided to the isolation zone 40 to safely remove particles, droplets, etc., as described below. In some optional embodiments, systems of the present disclosure can further include a UV output halo or channel as known in the art that is placed over the patient 100; UV light is directed toward the isolation region 40 to sterilize the contained air.

Where provided, the optional access flap(s) 70 can facilitate a clinician interfacing with the patient 100 within the isolation region 40 by simply inserting his/her hand through the flap 70. By way of non-limiting example, a clinician can perform one or more steps of a drug-induced sleep endoscopy procedure via the flap(s) 70, such as jaw/mandible thrust, etc.

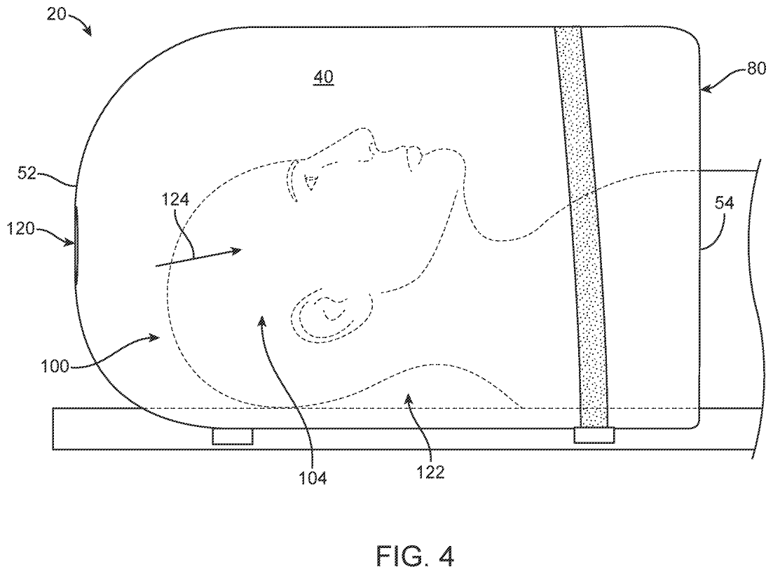

In some embodiments, airflow or pressure is established within the isolation region 40, serving to carry or evacuate respiratory droplets or particles entrained in the airflow away from the patient 100 in a safe manner. For example, and with reference to FIG. 4, the shield device 20 can optionally include an airflow connector or inlet 120, for example at the top end 52. The airflow inlet 120 can be connected to a source of positive pressure (not shown), such as a blower, that delivers a constant flow of gas (e.g., air) into the isolation region 40. Additionally or alternatively, an airflow inlet 122 (referenced generally) can be established at a location behind the patient's head 104. Regardless, the constant, positive pressure airflow travels toward the bottom end 54 (as indicated by arrow 124), entraining particles, droplets, etc., within the isolation region 40 (e.g., particles or droplets exhaled by the patient 100). The bottom end 54 serves as an airflow outlet from the isolation region 40, with the optional filter 80 capturing and removing the particles, droplets, etc., from the airflow as the airflow exits the shield device 20 to the surrounding environment. Alternatively, the shield device 20 can be configured to provide an airflow connector or outlet at the bottom end 54 that can be connected to a source of negative pressure (e.g., a conventional hospital suction canister). The constant, negative pressure airflow draws air and any entrained particles, droplets, etc., through the isolation region 40 (in a general direction of the airflow path 124). The so-evacuated airflow can be passed through a filter before being released to the environment. With these and other negative pressure-type installations, one or more air vents or inlets can be formed through the barrier 30, for example at or near the top end 52.

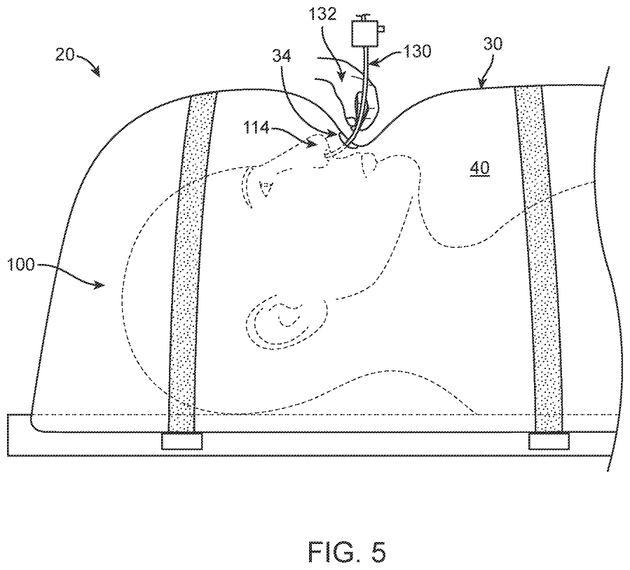

Regardless of whether positive or negative pressure airflow is provided to the isolation region 40, various medical procedures can be performed on the patient with the shield device 20 in place. For example, as shown in FIG. 5, the access port 34 facilitates placement of an endoscope or other medical device 130 into the patient's airways (e.g., via the nose 114). A clinician 132 grasps the medical device 130 from outside of the shield device 20 and inserts the medical device 130 through the access port 34. A construction of the access port 34 is such that an airtight, or nearly airtight, seal is formed and maintained about the so-inserted medical device 130, thereby preventing release of particles, droplets, etc., from the isolation region 40. As reflected by FIG. 5, the barrier 30 is flexible in some embodiments, and thus does not impede the clinician 132 in manipulating the medical device 130 relative to the patient 100 such that the clinician 132 can readily position and move the medical device 130 as desired (e.g., insertion into the nose 114). Upon completion of the procedure, the medical device 130 can be removed from the patient 100 and the shield device 20 without compromising an integrity of airflow within the isolation region 40.

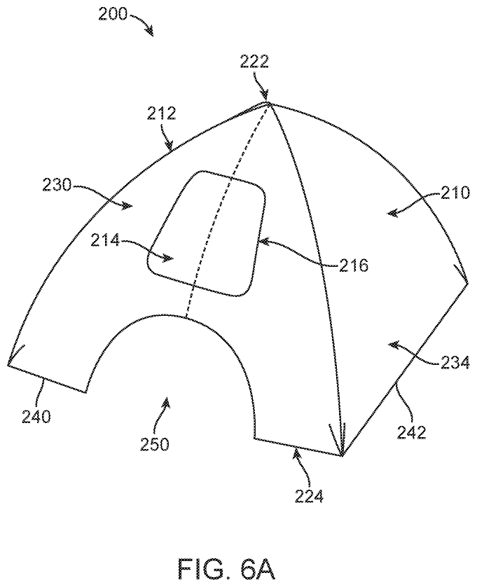

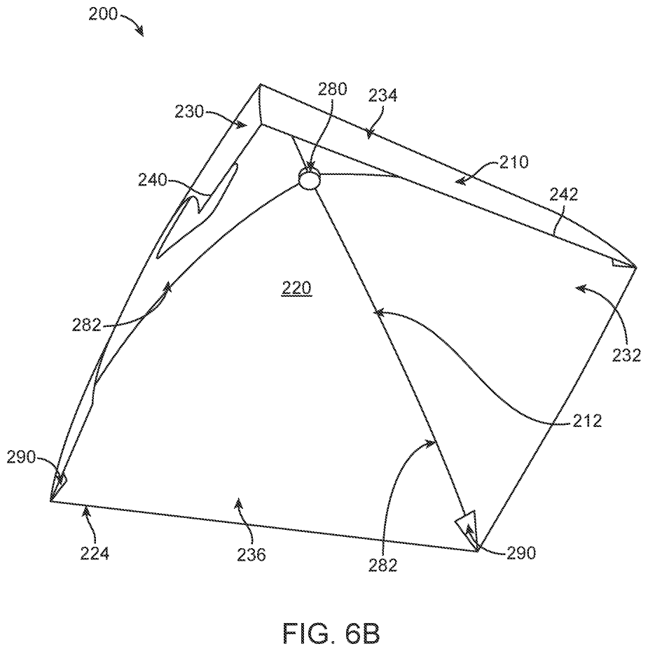

Another example of a shield device 200 in accordance with principles of the present is disclosure is shown in FIGS. 6A and 6B. The shield device 200 can have one or more of the features and/or can be used with one or more of the procedures described with respect to FIGS. 1A-5. The shield device 200 includes a barrier or shell 210, a frame 212 (referenced generally), and a flap or drape 214 removably covering an access port 216 (referenced generally). Details on the various components are provided below. In general terms, in the deployed state of FIGS. 6A and 6B, the frame 212 retains the barrier 210 in a shape conducive for placement over a head and neck of a reclined patient, creating an isolation region 220 (reference generally) that encompasses at least the patient's mouth and nose. For example, a shape of the shield device 200 can define a top 222 opposite a bottom 224. The top 222 can be considered closed, whereas the bottom 224 is open to the isolation region 220. The shield device 200 can thus be placed over the head of a supine patient, with the bottom 224 abutting a surface on which the patient is lying. All, or virtually all, particles, droplets, etc., emanating from the patient's mouth and nose (e.g., respiratory excretions) are contained within the isolation region 220 and are prevented from escaping into the surrounding, outside environment. When the flap 214 is lifted or otherwise displaced to expose the access port 216, the access port 216 facilitates placement of a medical device within the airway of a patient otherwise stationed within the isolation region 220 in a manner that does not affect containment of respiratory excretions, such as, for example, placement of an endoscope into the patient's nose or mouth. The shield device 210 permits performance of various respiratory-related procedures on a patient, for example drug-induced sleep endoscopy as described above, with minimal or no risk of aerosolized particles or droplets from the patient's respiratory system coming in contact with a care provider or escaping into the surrounding environment.

A shape of the barrier 210, at least in the deployed state of the shield device 200, can be dictated by the frame 212, and in some embodiments can be viewed as defining a plurality of barrier panels, such as a front panel 230, a rear panel 232, and opposing, first and second side panels 234, 236. The panels 230-236 commonly extend from the top 222 to a corresponding bottom edge (e.g., a bottom edge 240 of the front panel 230 and a bottom edge 242 of the first side panel 234 are labeled in FIGS. 6A and 6B). In some embodiments, the barrier panels 230-236 are collectively formed as a continuous, homogenous sheet of material or film. In other embodiments, the panels 230-236 can be separately formed and subsequently assembled to one another and/or the frame 212. Regardless, a material and construction of the barrier 210 can assume various forms, and in some embodiments comprises a flexible sheet, or layered sheets, of material that is impervious to air and bacterial sub-particles. In some embodiments, at least the front panel 230 is transparent or substantially transparent (i.e., within 10 percent of truly transparent). In other embodiments, two or more or all of the panels 230-236 are transparent or substantially transparent. In some non-limiting examples, the barrier 210 (e.g., the barrier material of each of the panels 230-236) can be a polyethylene terephthalate (PET) film (e.g., 0.003 inch thickness) or other substantially transparent polymer film.

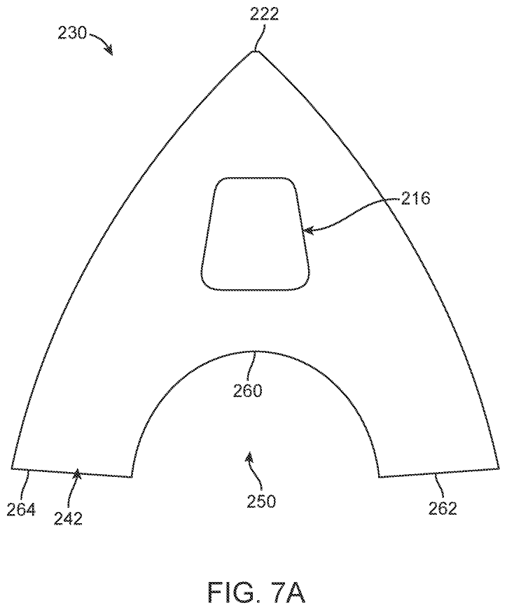

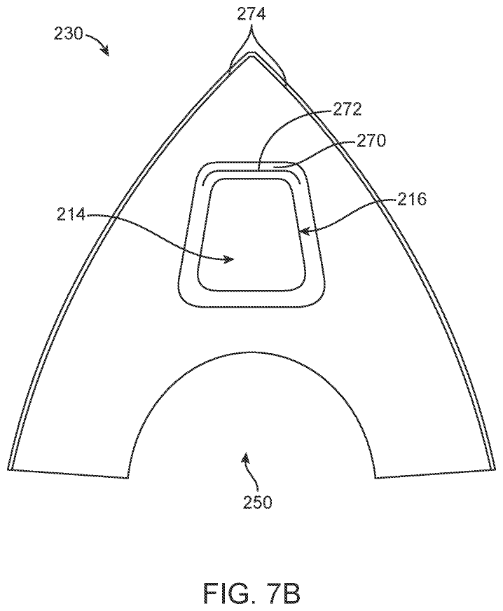

The front panel 230 is shown in isolation in FIG. 7A. An opening or recess 250 is defined by the bottom edge 242. For example, the bottom edge 242 can be viewed or defined as having a central segment 260 and opposing, first and second side segments 262, 264. The opposing side segments 262, 264 are contiguous with the bottom edge of the corresponding side panel (e.g., with additional reference to FIGS. 6A and 6B, the first side segment 262 of the bottom edge 242 of the front panel 230 is contiguous with the bottom edge 244 of the first side panel 234). The bottom edge 242 extends toward the top 222 from each of the side segments 262, 264 to generate the opening 250. In some embodiments, a shape of the central segment 260 can be, or can be akin to, a semi-circle as reflected by FIG. 7A, although other shapes (regular or irregular) are also acceptable. Regardless, the opening 250 is sized and shaped (e.g., height, width, diameter, etc.) to received, or for placement over, a neck or other anatomy of the upper body of a human adult. Some non-limiting example dimensions are provided below.

The access port 216 is formed through a thickness of the front panel 230 at a location between the top 222 and the bottom edge 242. A perimeter shape of the access port 216 can vary from the shapes implicated by FIG. 7A, and various dimensions can be employed. In general terms, the access port 216 is sized and shaped to facilitate performance of an expected medical procedure. For example, a size and shape of the access port 216 can be sufficient for passage of a medical device (e.g., endoscope), a caregiver's hand(s), etc. In some non-limiting examples, the access port 216 can have a maximum height on the order of 1-5 inches, alternatively on the order of 2-4 inches, and a maximum width on the order of 1-5 inches, alternatively on the order of 2-4 inches. In one example, the access port 216 has a maximum height of approximately 2.85 inches and a maximum width of approximately 2.70 inches, although other dimensions are equally acceptable.

FIG. 7B illustrates the flap 214 assembled to the front panel 230. The flap 214 can have the same material and construction as that of the barrier 210, and thus is shown in FIG. 7B as being substantially transparent. Other constructions are also acceptable. Regardless, a size and shape of the flap 214 corresponds with that of the access port 216, with the flap 214 being configured to encompass or cover an entirety of the access port 216 in the closed condition of FIG. 7B. The flap 214 can be assembled to the front panel 230 in various manners that promote the flap 214 naturally assuming the closed condition and affording a user to readily displace the flap 214 from the closed condition. For example, in some embodiments, an upper edge section 270 of the flap is secured to a material of the front panel 230 (e.g., bonding, ultrasonic welding, repositionable adhesive, etc.) along a line of securement 272 at a location above the access port 216 (relative to the orientation of FIG. 7B), whereas a remainder of the flap 214 is free of attachment to the front panel 230. With this optional construction, the flap 214 will naturally fall or drape over the access port 216 in the upright orientation of the shield device 200 (FIG. 6A) while edges other than the upper edge section 270 can be manually moved or lifted away from the front panel 230 to open the access port 216. As a point of reference, FIG. 7B further identifies at 274 optional, additional lines of securement (e.g., bonding, ultrasonic welding) at which the front panel 230 is attached to a corresponding one of the side panels 234, 236 (FIG. 6A).

With additional reference to FIGS. 6A and 6B, apart from the opening 250, the perimeter shape of the front panel 230 reflected by the flat, plan view of FIG. 7A can be utilized with each of the remaining panels 232-236. Thus, each of the panels 230-236 can, in flat form, have a triangular-like shape, tapering in width from the corresponding bottom edge to the top 222. As implicated by FIG. 7A, side edges of the triangular-like shape can be curved. Regardless, the triangular-like shape of the panels 230-236 is, upon final assembly, conducive to forming a tent-like shape or structure by the frame 212 in the deployed state. In some examples, each of the panels 230-236 can have, in flat form, a height on the order of 9-13 inches, alternatively 10-12 inches, alternatively approximately 10.74 inches; and a maximum width (e.g., linear distance along the corresponding bottom edge) on the order of 10-14 inches, alternatively 11-13 inches, alternatively approximately 12.15 inches. With these and related embodiment, an outer dimension (e.g., diameter) of the opening 250 can be on the order of 4-8 inches, alternatively 5-7 inches, alternatively approximately 6.13 inches. A wide variety of other dimensions or geometries are also acceptable.

The frame 212 can assume various forms conducive to supporting the barrier 210 in the deployed state, and optionally collapsible to a collapsed state. In some examples, the frame 212 can include a hub 280 and support arms 282. The support arms 282 can each be a thin body with shape resiliency, for example a spring steel wire or the like. The support arms 282 are attached to and extend from hub 280, and are biased to, or can naturally assume, the shape reflected by FIGS. 6A and 6B.

The barrier 210 can be assembled to the frame 212 in various manners. In some examples, each of the panels 230-236 are attached to the hub 280 to create the top 220. The support arms 282 extend from the hub 280 along respective ones of the lines of intersection or corners between adjacent panels 232-236. The support arms 282 can be connected to the barrier 210 opposite the hub 280. For example, and as best shown in FIG. 6B, a pocket 290 can be provided along an interior of the barrier 210 at the corner between an adjacent pair of the panels 232-236 near or at the bottom 224. Each of the pockets 290 are configured to receive a free end of a corresponding one of the support arms 282. The free end of the support arm 282 can be removably placed into the corresponding pocket 290, or a more permanent attachment can be provided. Regardless, with this optional construction, the support arms 282 each exert an expansion force onto the corresponding corner of the barrier 210, forcing the barrier 210 to the expanded state as shown. Other assembly techniques can also be employed.

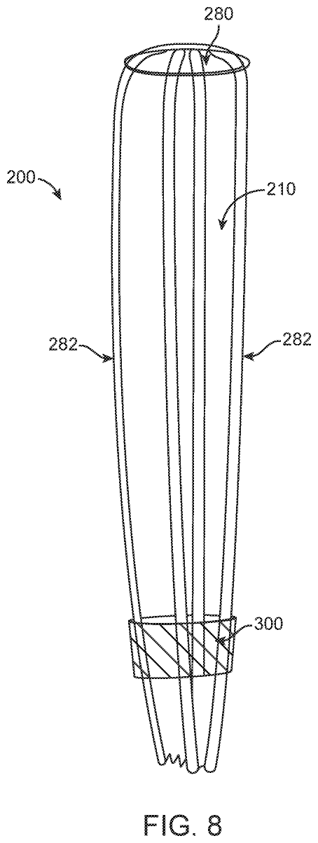

In some embodiments, a construction of the frame 212 along with a flexible nature of the barrier 210 renders the shield device 200 collapsible from the deployed state of FIGS. 6A and 6B to a collapsed state that can be more conducive to shipping and/or storage. FIG. 8 is one example of a collapsed state of the shield device 200. As shown, the support arms 282 have been forced toward one another, pivoting at the hub 280. The barrier 210 readily folds onto itself with inward deflection of the support arms 282. A constraint 300 (e.g., band, ring, etc.) can be placed over or about the shield device 200, serving to hold the shield device 200 in the collapsed state. The frame 212 is, in some embodiments, configured to self-expand upon removal of the constraint 300 (and any other packaging), causing the shield device 200 to self-revert to the deployed state of FIGS. 6A and 6B.

The shield devices and related systems and methods of use provide a marked improvement over previous designs. Unlike a conventional oronasal mask or nasal mask fitted with a bronchoscopy elbow, the shield devices of the present disclosure creates a flexible, non-claustrophobic barrier about the patient while facilitating performance of a desired respiratory airway-related procedures, such as drug-induced sleep endoscopy. The shield devices of the present disclosure will not force the patient's mouth to stay shut (which may not otherwise be a natural sleeping posture and could confound the findings of an airway assessment, especially if the action on the temporomandibular joint (TMJ) is such that the mandible is displaced posteriorly). Moreover, the shield devices of the present disclosure avoid circumstances where by a patient undergoing a particular procedure, such as a sleep assessment, is otherwise caused enough discomfort by a claustrophobic mask such that more sedative agent is required that could over-sedate the patient; this, in turn, could lead to substantial reductions in the positive and negative predictive values of a procedure for determining candidacy for upper air stimulation. Further, the shield devices of the present disclosure do not overtly limit the ability of a practitioner to freely execute steps of a particular procedure (e.g., drug-induced sleep endoscopy) that otherwise considered dangerous for risk of respiratory illness transmissivity. For example, the shield devices of the present disclosure can facilitate performance of desired actions as part of a sleep study with drug-induced sleep endoscopy, such as a jaw-thrust (e.g., Esmarch) maneuvers, adjusting the level of the endoscope (e.g., while assessing multiple levels of the airway's vulnerable Starling Resistor segments from the genu of the velopharynx superiorly through to the epiglottis and arytenoids inferiorly), etc.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. A shield device for isolating respiratory excretions of a patient, the shield device comprising:
a barrier;
at least one support arm maintaining the barrier in a deployed state having size and shape accommodating a head of a patient, wherein the barrier establishes an isolation region in the deployed state; and
an access port formed through the barrier and adapted to permit sealed access to the isolation region from an exterior of the shield device by a medical device.

2. The shield device of claim 1, wherein the barrier is a flexible material.

3. The shield device of claim 2, wherein the barrier is a polymer material.

4. The shield device of claim 2, wherein the barrier is impervious to air.

5. The shield device of claim 2, wherein the barrier is impervious to airborne pathogens.

6. The shield device of claim 2, wherein at least a segment of the barrier is substantially transparent.

7. The shield device of claim 1, wherein the at least one support arm defines a dome-like shape in the deployed state.

8. The shield device of claim 1, wherein the barrier defines a top end opposite a bottom end, and further wherein the top end is closed relative to the isolation region.

9. The shield device of claim 1, wherein the access port is configured for sealed insertion of an endoscope.

10. The shield device of claim 1, wherein the barrier further defines at least one flap configured for passage of a clinician's hand.

11. The shield device of claim 1, wherein the shield device is configured for use with a drug-induced sleep endoscopy procedure.

12. The shield device of claim 1, wherein the at least one support arm is provided as part of a frame that further includes a hub from which at least two of the support arms extend, and further wherein the barrier is secured to the hub to define a top of the shield device.

13. The shield device of claim 12, wherein the barrier includes first, second, third and fourth barrier panels, and further wherein adjacent ones of the barrier panels are secured to one another at a corresponding corner.

14. The shield device of claim 13, wherein the frame is configured to self-expand from a collapsed state to the deployed state.

15. A method of performing a medical procedure on airways of a patient, the method comprising:
deploying a shield device over a head of patient, the shield device including a barrier supported by at least one support arm and an access port carried by the barrier;
wherein following the step of deploying, at least a mouth and nose of the patient are contained within an isolation region defined by the shield device;
inserting a medical device through the access port and into an airway of the patient;
wherein respiratory excretions from the patient are contained within the isolation region.

16. The method of claim 15, wherein the patient is supine following the step of deploying the shield device.

17. The method of claim 15, wherein prior to the step of deploying, the patient is supine on a support.

18. The method of claim 17, wherein the step of deploying includes articulating the shield device relative to the support.

19. The method of claim 15, further comprising interfacing with the patient by a caregiver through a flap formed in the barrier.

20. The method of claim 15, wherein the medical procedure includes a drug-induced sleep endoscopy procedure.
